(19) United States
(12) Patent Application Publication
(10) Pub. No.: US 2008/0214394 A1
(43) Pub. Date: Sep. 4, 2008

(54) N-(4-Pyridyl)Methylsulfonamides For Combating Arthropodal Pests

(75) Inventors: Michael Puhl, Lampertheim (DE); Christopher Koradin, Ludwigshafen (DE); Joachim Rheinheimer, Ludwigshafen (DE); Wassilios Grammenos, Ludwigshafen (DE); Jan Klaas Lohmann, Mannheim (DE); Ernst Baumann, Dudenhofen (DE); Wolfgang von Deyn, Neustadt (DE); Jurgen Langewald, Mannheim (DE); Norbert Gotz, Worms (DE); Douglas D. Anspaugh, Apex, NC (US); Deborah L. Culbertson, Fuquay Varina (IT); Henry Van Tuyl Cotter, Raleigh, NC (US); Hassan Olumi-Sadeghi, Raleigh, NC (US)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/908,744

(22) PCT Filed: Mar. 15, 2006
(86) PCT No.: PCT/EP2006/060752

§ 371 Date: Sep. 14, 2007

Related U.S. Application Data
(60) Provisional application No. 60/662,411, filed on Mar. 16, 2005.

(30) Foreign Application Priority Data
May 30, 2005 (EP) 05011598.9

(51) Int. Cl. A01N 43/40; C07D 215/36; C07D 401/12; A61K 31/44

(52) U.S. Cl. 504/100; 514/357; 546/172; 546/280.4

(57) ABSTRACT

N-(4-Pyridyl)methylsulfonamides for combating arthropodal pests The present invention relates to the use of N-(4-pyridyl)methylsulfonamides of the formula for combating arthropodal pests (harmful arthropodes) and for protecting materials against infestation and/or destruction by said pests: (I) where the substituents are as follows: R¹ is hydrogen, C₁-C₄-alkyl, C₁-C₄-alkoxy, C₂-C₄-alkenyl, C₂-C₄-alkynyl or benzyl; R², R³, R⁴, R⁵ independently of one another are hydrogen, halogen, C₁-C₄-alkyl, C₁-C₄-alkoxy, C₁-C₄-haloalkoxy or C₁-C₄-haloalkyl; R² and R³ or R⁴ and R⁵ together with the carbon atoms to which they are attached may also form a condensed 5- or 6-membered hydro-carbon ring, it being possible for the hydrocarbon ring to carry one or two groups R^{2′}, R^{3′}, R^{2′}, R^{3′} independently of one another are halogen, C₁-C₄-alkyl, C₁-C₄-alkoxy, halomethoxy or halomethyl; X is a cyclic radical selected from phenyl, naphthyl and five- or six-membered saturated, partially unsaturated or aromatic heterocycles, the heterocycle being attached to the sulfur atom via a carbon atom and containing 1, 2 or 4 heteroatoms selected from the group consisting of O, N and S, where the cyclic radical X may carry 1, 2, 3 or 4 substituents R^{a}.

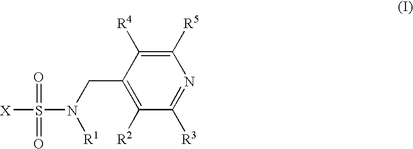

The present invention relates to the use of N-(4-pyridyl)methylsulfonamides for combating arthropodal pests (harmful arthropodes) and for protecting materials against infestation and/or destruction by said pests.

In spite of commercial pesticides available today, damage to crops, both growing and harvested, the damage of non-living material, in particular cellulose based materials such as wood or paper, caused by arthropodal pests still occur, either because the action of know compounds is unsatisfactory or because target pest have acquired resistance against known actives.

Co-pending application WO 05/033081 discloses N-(4-pyridyl)methylsulfonamides fungicidal plant protection agents. No mention is made of their insecticidal activity.

JP 63-227552 discloses N,N-disubstituted 2-fluoroethylamines of the formula

R¹R²N—CH₂—CH₂—F

wherein R¹ is phenyl, phenylalkyl, pyridyl or pyridylalkyl and R² is H, (halo)alkyl, alkanoylalkyl, (halo)alkanoyl, alkoxycarbonyl, phenylalkanoyl, phenylsulfonyl, N-alkylcarbamoyl, a 5-membered or 6-membered heterocyclic ring, phenyl, benzoyl or R¹ and R² together with the nitrogen form a carbazole ring or a phenothiazine ring. The compounds are described to be effective against insects.

Based on this, there is ongoing need to provide compounds which are useful for combating harmful arthropodes such as insects and arachnids. It is desirable that the compounds have an improved action and/or a broader activity spectrum against harmful arthropodes.

Accordingly we have found that this object is achieved by N-(4-pyridyl)methyl-sulfonamides of the formula I as defined herein.

Therefore, the present invention relates to the use of N-(4-pyridyl)methylsulfonamides of the formula I

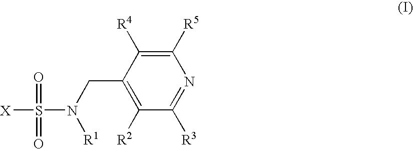

where the substituents are as follows:
R¹ is hydrogen, C₁-C₄-alkyl, C₁-C₄-alkoxy, C₂-C₄-alkenyl, C₂-C₄-alkynyl or benzyl;
R², R³, R⁴, R⁵ independently of one another are hydrogen, halogen, C₁-C₄-alkyl, C₁-C₄-alkoxy, C₁-C₄-haloalkoxy or C₁-C₄-haloalkyl;
   R² and R³ or R⁴ and R⁵ together with the carbon atoms to which they are attached may also form a condensed 5- or 6-membered hydro-carbon ring, it being possible for the hydrocarbon ring to carry one or two groups R^{2′}, R^{3′},
   R^{2′}, R^{3′} independently of one another are halogen, C₁-C₄-alkyl, C₁-C₄-alkoxy, halomethoxy or halomethyl;
X is a cyclic radical selected from phenyl, naphthyl and five- or six-membered saturated, partially unsaturated or aromatic heterocycles, the heterocycle being attached to the sulfur atom via a carbon atom and containing 1, 2 or 4 heteroatoms selected from the group consisting of O, N and S, where the cyclic radical X may carry 1, 2, 3 or 4 substituents R^{a}:
   R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl or
      phenyl or phenoxy, where the phenyl ring in the last two mentioned radicals may carry 1, 2, 3, 4 or 5 groups R^{b}:
      R⁶ is C₁-C₄-alkyl,
      R⁷ is C₁-C₈-alkyl, benzyl, C₂-C₄-alkenyl, C₁-C₄-haloalkyl, C₂-C₄-haloalkenyl, C₂-C₄-alkynyl or C₂-C₄-haloalkynyl; and
      R^{b} is halogen, C₁-C₄-alkyl, C₁-C₄-alkoxy, C₁-haloalkyl, phenyl, optionally substituted with halogen, or haloalkoxy;
      two radicals R^{a} or two radicals R^{b}, together with two adjacent ring members of the phenyl ring to which they are attached may form a hydrocarbon ring which may be substituted by one or more of the abovementioned groups R^{a} or R^{b},
         with the exception of compounds wherein X and R^{a} together form an optionally substituted biphenyl, and R², R³, R⁴ and R⁵ independently of one another are hydrogen, halogen, C₁-C₄-alkyl, C₁-C₄-alkoxy, C₁-C₄-haloalkoxy or C₁-C₄-haloalkyl; and the N-oxides and the agriculturally and veterinarilly acceptable their salts of compounds of formula I, for combating harmful arthropodes.

N-(4-Pyridyl)methylsulfonamides of the formula I are known from PCT/EP 04/010124. Unsubstituted 4-pyridinylmethanesulfonamides are known from EP-A 206 581 and Lieb. Ann. Chem. 641 (1990). The compounds described in these publications mentioned are suitable for controlling harmful fungi.

Due to their excellent activity, the compounds of the general formula I can be used for controlling arthropodal pests. The compounds of the formula I are in particular useful from combating insects. Likewise the compounds of the formula I and their salts are in particular useful from combating arachnids.

The term “combating” as used herein comprises controlling, i.e. killing of pests and also protecting plants, non-living materials or seeds from an attack or infestation by said pests.

Accordingly, the invention further provides compositions for combating such pests, preferably in the form of directly sprayable solutions, emulsions, pastes, oil dispersions, powders, materials for scattering, dusts or in the form of granules, which comprises a pesticidally effective amount of at least one compound of the general formula I or at least a salt thereof and at least one carrier which may be liquid and/or solid and which is preferably agronomically acceptable, and/or at least one surfactant.

Furthermore, the invention provides a method for combating such pests, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a compound of the general formula I as defined herein or a salt thereof.

The invention provides in particular a method for protecting crops, including seeds, from attack or infestation by arthropodal pests, said method comprises contacting a crop with a pesticidally effective amount of at least one compound of formula I as defined herein or with a salt thereof.

The invention also provides a method for protecting non-living materials from attack or infestation by the aforementioned pests, which method comprises contacting the non-living material with a pesticidally effective amount of at least one compound of formula I as defined herein or with a salt thereof.

Suitable compounds of the general formula I encompass all possible stereoisomers (cis/trans isomers, enantiomers) which may occur and mixtures thereof. Stereoisomeric centers are e.g. the carbon and nitrogen atom of the —C(R⁶)═NOR⁷ moiety as well as asymmetric carbon atoms in the radicals R^{a}, R¹, R², R³, R⁴ and/or R⁵ etc. The present invention provides both the pure enantiomes or diastereomers or mixtures thereof, the pure cis- and trans-isomers and the mixtures thereof. The compounds of the general formula I may also exist in the form of different tautomers. The invention comprises the single tautomers, if seperable, as well as the tautomer mixtures.

Salts of the compounds of the formula I are preferably agriculturally acceptable salts. They can be formed in a customary method, e.g. by reacting the compound with an acid of the anion in question if the compound of formula I has a basic functionality or by reacting an acidic compound of formula I with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae Ia and Ib with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

halogen: fluorine, chlorine, bromine and iodine;
alkyl: saturated straight-chain or branched hydrocarbon radicals having 1 to 4, 6 or 8 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2 or 4 carbon atoms (as mentioned above), where in these groups some or all of the hydrogen atoms may be replaced by halogen atoms as mentioned above; in particular, C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 6 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- to ten-membered saturated, partially unsaturated or aromatic heterocycle which contains one to four heteroatoms from the group consisting of O, N and S:
5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example, 2-thienyl, 3-thienyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O;
   alkenylene: divalent unbranched chains of 4 or 6 CH groups which are linked by conjugated C═C double bonds, for example CH═CH or CH═CH—CH═CH.

Condensed 5- or 6-membered hydrocarbon ring means a hydrocarbon ring which shares two adjacent carbon atoms with another ring, examples being cylopentane, cyclopentene, cyclohexane, cyclohexene and benzene.

With a view to the intended use of the sulfonamides of the formula I, particular preference is given to the following meanings of the substituents, in each case on their own or in combination:

The invention preferably provides compounds of the formula I, in which R¹ is hydrogen, methyl, methoxy, ethoxy, allyl or propargyl, in particular hydrogen or methyl.

Preference is likewise given to compounds of the formula I, in which R², R³, R⁴ and R⁵ independently of one another are hydrogen, methyl, ethyl, fluorine, chlorine, CF₃, OCF₃ or OCHF₂.

One preferred embodiment of the invention relates to the use of compounds of the formula I in which at least one, in particular one or two, groups selected from the group consisting of R², R³, R⁴ and R⁵ are not hydrogen.

Preference is likewise also given to compounds of the formula I, wherein the radicals R² and R³ together with the atoms to which they are bound form a condensed benzene ring, i.e. R² and R³ together form a bivalent radical —CH═CH—CH═CH—, wherein one or two of the hydrogen atoms may be replaced by the radicals R^{2′} and/or R^{3′}.

Another preferred embodiment of the invention relates to the use of compounds of the formula I in which each of the radicals R², R³, R⁴ and R⁵ are hydrogen. In this embodiment, preference is given to compounds, wherein X carries at least one radical R^{a}, which is different from hydrogen. Amongst these preference is given to compounds I, wherein one of the radicals R^{a} is a radical —C(R⁶)═NOR⁷. In this embodiment X is preferably phenyl, which, in particular, carries a radical in the 4-position, or thienyl, in particular 2-thienyl, which may carry a radical R^{a} in the 5-position.

Further preferred embodiments of the formula I are in each case per se compounds of the formulae I.1 to I.7 where the variables X and R¹ are as defined for formula I, m and k are each independently 0 or 1 and wherein the variables R², R³, R⁴, R⁵ have the aforementioned meanings, except for hydrogen:

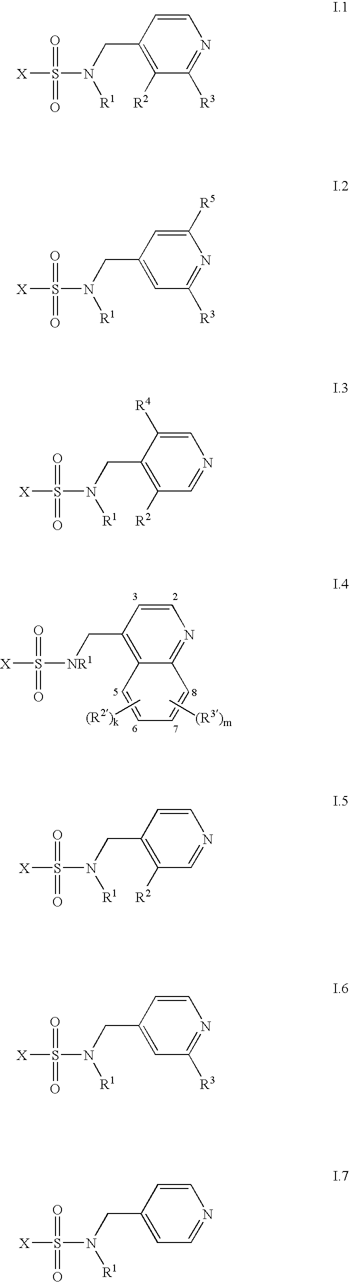

Among compounds of the formula I.4 preference is given to those in which the groups R^{2′} and R^{3′} (if present) are located in the 6- and/or 7-position.

Moreover, preference is given to the new compounds of formula IA.4′

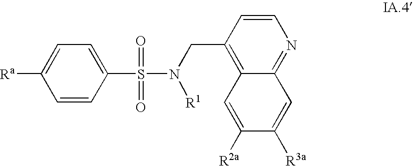

wherein R^{2a} and R^{3a} are both hydrogen, methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Preference is given to compounds of the formula I, wherein X is a phenyl ring which is unsubstituted or carries 1, 2 or 3 radicals R^{a}. Amongst these, compounds are preferred wherein phenyl carries a radical R^{a} in the para-position.

Likewise, preference is given to compounds I, wherein X is an aromatic heterocycle, in particular a thiophene ring, more preferably 2-thienyl. The thiophene ring may be unsubstituted or may carry 1, 2 or 3 radicals R^{a} as defined above. Amongst those, preference is given to compounds I, wherein X is 2-thienyl, which carries a radical R^{a} in the 5-position. R^{a} has preferably one of the following meanings: C(R⁶)═NOR⁷, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy, tert-butoxy, chloromethyl, dichloromethyl, trichloromethyl, fluoromethyl, difluoromethyl, trifluoromethyl, dichlorofluoromethyl, chlorodifluoromethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, trichloromethoxy, fluoromethoxy, difluoromethoxy, trifluoromethoxy, chlorodifluoromethoxy, 2,2-difluoroethoxy, 2,2,2-trifluoroethoxy, 2,2,2-trichloroethoxy and pentafluoroethoxy, chloro, bromo, phenyl, 4-phenyl-phenyl, 4-chlorophenyl, 4-bromophenyl, 4-methylphenyl, 4-methoxyphenyl, 4-ethylphenyl, 4-(n-propyl)phenyl, 4-(1-methylethyl)phenyl, 4-tert.-butylphenyl, 4-trifluorophenyl, 4-trifluormethoxyphenyl.

A particularly preferred embodiment of R⁶ is methyl; R⁷ is preferably methyl, ethyl, n-propyl, isopropyl, n-butyl, isobutyl, allyl or propargyl, it being possible for the groups R⁷ to be halogenated.

Particular preference is given to compounds of the formula I in which X is a phenyl ring which carries exactly one group R^{a} in the para-position; these compounds correspond to the formula IA:

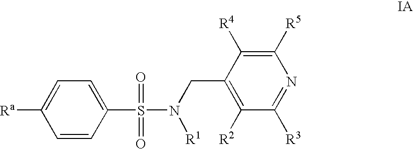

Particular preference is given to compounds of the formula IA, in which R^{a} has the following meanings: C(R⁶)═NOR⁷, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy, tert-butoxy, chloromethyl, dichloromethyl, trichloromethyl, fluoromethyl, difluoromethyl, trifluoromethyl, dichlorofluoromethyl, chlorodifluoromethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, trichloromethoxy, fluoromethoxy, difluoromethoxy, trifluoromethoxy, chlorodifluoromethoxy, 2,2-difluoroethoxy, 2,2,2-trifluoroethoxy, 2,2,2-trichloroethoxy and pentafluoroethoxy.

Particular preference is also given to compounds of the formula I in which X is a 2-thienyl ring which carries a group R^{a} in the 5-position; these compounds correspond to the formula IB:

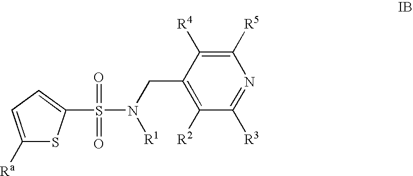

Likewise, particular preference is given to compounds of the formula IB, in which R^{a} has the following meanings: C(R⁶)═NOR⁷, methyl, ethyl, n-propyl, isopropyl, n-butyl, isobutyl, sec-butyl, tert-butyl, methoxy, ethoxy, n-propoxy, isopropoxy, n-butoxy, isobutoxy, sec-butoxy, tert-butoxy, chloromethyl, dichloromethyl, trichloromethyl, fluoromethyl, difluoromethyl, trifluoromethyl, dichlorofluoromethyl, chlorodifluoromethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, trichloromethoxy, fluoromethoxy, difluoromethoxy, trifluoromethoxy, chlorodifluoromethoxy, 2,2-difluoroethoxy, 2,2,2-trifluoroethoxy, 2,2,2-trichloroethoxy and pentafluoroethoxy, chloro, bromo, phenyl, 4-phenyl-phenyl, 4-chlorophenyl, 4-bromophenyl, 4-methylphenyl, 4-methoxyphenyl, 4-ethylphenyl, 4-(n-propyl)phenyl, 4-(1-methylethyl)phenyl, 4-tert.-butylphenyl, 4-trifluorophenyl, 4-trifluormethoxyphenyl.

Moreover, particular preference is given to the new compounds of the formula IB wherein R², R³, R⁴ and R⁵ are hydrogen, R¹ is hydrogen or methyl, and R^{a} is selected from phenyl, 4-phenyl-phenyl, 4-methyl-phenyl and 5-ethyl-phenyl.

Also, particular preference is given to the new compounds of formula IB.1

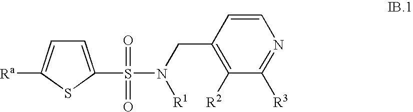

wherein R² and R³ are both methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Besides, particular preference is given to the new compounds of formula IB.2

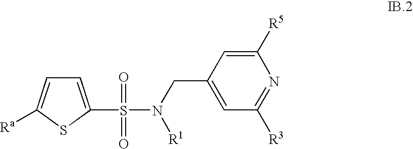

wherein R³ and R⁵ are both methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Moreover, particular preference is given to the new compounds of formula IB.3

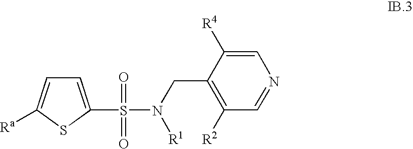

wherein R² and R⁴ are both methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Also, particular preference is given to the new compounds of formula IB.4′

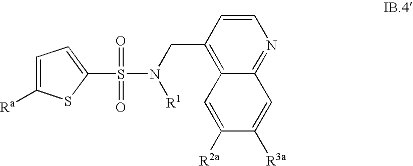

wherein R^{2a} and R^{3a} are both hydrogen, methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Besides, particular preference is given to the new compounds of formula IB.5

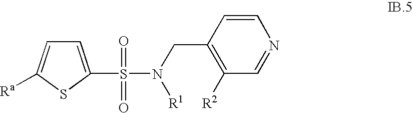

wherein R² is methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

Also, particular preference is given to the new compounds of formula IB.6

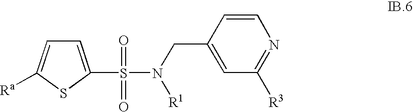

wherein R³ is methyl, fluorine, chlorine, methoxy, or trifluoromethoxy, R¹ is hydrogen or methyl, and R^{a} is selected from phenyl, 4-tert.-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl and 4-isopropyl-phenyl.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula IA, in which R², R³, R⁴ and R⁵ are hydrogen and the combination of R¹ and R^{a} corresponds for each compound to one row of Table A selected from the rows A-1 to A-78, A-81, A-82 and A-85 to A-88

### Table 2

Compounds of the formula IA.1, in which R² and R³ are methyl and the combination of R¹ and R^{a} corresponds for each compound to one row of Table A selected from the rows A-1 to A-78, A-81, A-82 and A-85 to A-88

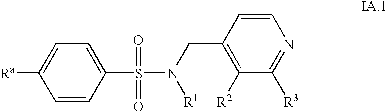

### Table 3

Compounds of the formula IA.1, in which R² and R³ are fluorine and the combination of R¹ and R^{a} corresponds for each compound to one row of Table A selected from the rows A-1 to A-78, A-81, A-82 and A-85 to A-88

### Table 4

Compounds of the formula IA.1, in which R² and R³ are chlorine and the combination of R¹ and R^{a} corresponds for each compound to one row of Table A selected from the rows A-1 to A-78, A-81, A-82 and A-85 to A-88

### Table 5

Compounds of the formula IA.1, in which R² and R³ are methoxy and the combination of R¹ and R^{a} corresponds for each compound to one row of Table A selected from the rows A-1 to A-78, A-81, A-82 and A-85 to A-88

### Table 6

Compounds of the formula IA.1, in which R² and R³ are trifluoromethoxy and the combination of R¹ and R^{a} corresponds for each compound to one row of Table A selected from the rows A-1 to A-78, A-81, A-82 and A-85 to A-88

### Table 7

Compounds of the formula IA.2, in which R³ and R⁵ are methyl and the combination of R¹ and R^{a} corresponds for each compound to one row of Table A selected from the rows A-1 to A-78, A-81; A-82 and A-85 to A-88

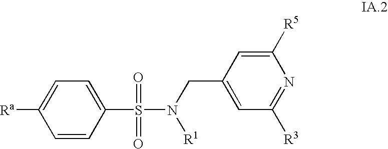

### Table 8

Compounds of the formula IA.2, in which R³ and R⁵ are fluorine and the combination of R¹ and R^{a} corresponds for each compound to one row of Table A selected from the rows A-1 to A-78, A-81, A-82 and A-85 to A-88

### Table 9

Compounds of the formula IA.2, in which R³ and R⁵ are chlorine and the combination of R¹ and R^{a} corresponds for each compound to one row of Table A selected from the rows A-1 to A-78, A-81, A-82 and A-85 to A-88

### Table 10

Compounds of the formula IA.2, in which R³ and R⁵ are methoxy and the combination of R¹ and R^{a} corresponds for each compound to one row of Table A selected from the rows A-1 to A-78, A-81, A-82 and A-85 to A-88

### Table 11

Compounds of the formula IA.2, in which R³ and R⁵ are trifluoromethoxy and the combination of R¹ and R^{a} corresponds for each compound to one row of Table A selected from the rows A-1 to A-78, A-81, A-82 and A-85 to A-88

### Table 12

Compounds of the formula IA.3, in which R² and R⁴ are methyl and the combination of R¹ and R^{a} corresponds for each compound to one row of Table A selected from the rows A-1 to A-78, A-81, A-82 and A-85 to A-88

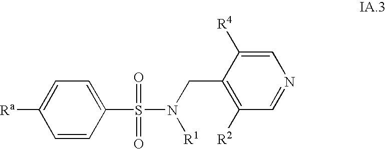

### Table 13

Compounds of the formula IA.3, in which R² and R⁴ are fluorine and the combination of R¹ and R^{a} corresponds for each compound to one row of Table A selected from the rows A-1 to A-78, A-81, A-82 and A-85 to A-88

### Table 14

Compounds of the formula IA.3, in which R² and R⁴ are chlorine and the combination of R¹ and R^{a} corresponds for each compound to one row of Table A selected from the rows A-1 to A-78, A-81, A-82 and A-85 to A-88

### Table 15

Compounds of the formula IA.3, in which R² and R⁴ are methoxy and the combination of R¹ and R^{a} corresponds for each compound to one row of Table A selected from the rows A-1 to A-78, A-81, A-82 and A-85 to A-88

### Table 16

Compounds of the formula IA.3, in which R² and R⁴ are trifluoromethoxy and the combination of R¹ and R^{a} corresponds for each compound to one row of Table A selected from the rows A-1 to A-78, A-81, A-82 and A-85 to A-88

### Table 17

Compounds of the formula IA.4′, in which R^{2a} and R^{3a} are hydrogen and the combination of R¹ and R^{a} corresponds for each compound to one row of Table A

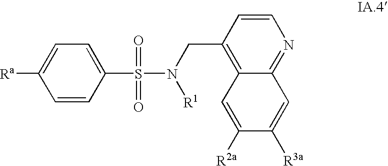

### Table 18

Compounds of the formula IA.4′, in which R^{2a} and R^{3a} are methyl and the combination of R¹ and R^{a} corresponds for each compound to one row of Table A

### Table 19

Compounds of the formula IA.4′, in which R^{2a} and R^{3a} are fluorine and the combination of R¹ and R^{a} corresponds for each compound to one row of Table A

### Table 20

Compounds of the formula IA.4′, in which R^{2a} and R^{3a} are chlorine and the combination of R¹ and R^{a} corresponds for each compound to one row of Table A

### Table 21

Compounds of the formula IA.4′, in which R^{2a} and R^{3a} are methoxy and the combination of R¹ and R^{a} corresponds for each compound to one row of Table A

### Table 22

Compounds of the formula IA.4′, in which R^{2a} and R^{3a} are trifluoromethoxy and the combination of R¹ and R^{a} corresponds for each compound to one row of Table A

### Table 23

Compounds of the formula IA.5, in which R² is methyl and the combination of R¹ and R^{a} corresponds for each compound to one row of Table A selected from the rows A-1 to A-78, A-81, A-82 and A-85 to A-88

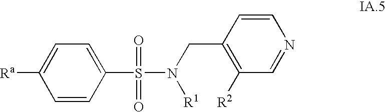

### Table 24

Compounds of the formula IA.5, in which R² is fluorine and the combination of R¹ and R^{a} corresponds for each compound to one row of Table A selected from the rows A-1 to A-78, A-81, A-82 and A-85 to A-88

### Table 25

Compounds of the formula IA.5, in which R² is chlorine and the combination of R¹ and R^{a} corresponds for each compound to one row of Table A selected from the rows A-1 to A-78, A-81, A-82 and A-85 to A-88

### Table 26

Compounds of the formula IA.5, in which R² is methoxy and the combination of R¹ and R^{a} corresponds for each compound to one row of Table A selected from the rows A-1 to A-78, A-81, A-82 and A-85 to A-88

### Table 27

Compounds of the formula IA.5, in which R² is trifluoromethoxy and the combination of R¹ and R^{a} corresponds for each compound to one row of Table A selected from the rows A-1 to A-78, A-81, A-82 and A-85 to A-88

### Table 28

Compounds of the formula IA.6, in which R³ is methyl and the combination of R¹ and R^{a} corresponds for each compound to one row of Table A selected from the rows A-1 to A-78, A-81, A-82 and A-85 to A-88

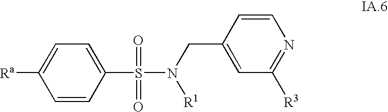

### Table 29

Compounds of the formula IA.6, in which R³ is fluorine and the combination of R¹ and R^{a} corresponds for each compound to one row of Table A selected from the rows A-1 to A-78, A-81, A-82 and A-85 to A-88

### Table 30

Compounds of the formula IA.6, in which R³ is chlorine and the combination of R¹ and R^{a} corresponds for each compound to one row of Table A selected from the rows A-1 to A-78, A-81, A-82 and A-85 to A-88

### Table 31

Compounds of the formula IA.6, in which R³ is methoxy and the combination of R¹ and R^{a} corresponds for each compound to one row of Table A selected from the rows A-1 to A-78, A-81, A-82 and A-85 to A-88

### Table 32

Compounds of the formula IA.6, in which R³ is trifluoromethoxy and the combination of R¹ and R^{a} corresponds for each compound to one row of Table A selected from the rows A-1 to A-78, A-81, A-82 and A-85 to A-88

### Table 33

Compounds of the formula IB, in which R², R³, R⁴ and R⁵ are hydrogen and the combination of R¹ and R^{a} corresponds for each compound to one row of Table A

### Table 34

Compounds of the formula IB.1, in which R² and R³ are methyl and the combination of R¹ and R^{a} corresponds for each compound to one row of Table A

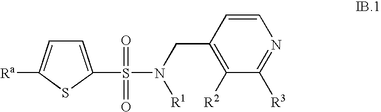

### Table 35

Compounds of the formula IB.1, in which R² and R³ are fluorine and the combination of R¹ and R^{a} corresponds for each compound to one row of Table A

### Table 36

Compounds of the formula IB.1, in which R² and R³ are chlorine and the combination of R¹ and R^{a} corresponds for each compound to one row of Table A

### Table 37

Compounds of the formula IB.1, in which R² and R³ are methoxy and the combination of R¹ and R^{a} corresponds for each compound to one row of Table A

### Table 38

Compounds of the formula IB.1, in which R² and R³ are trifluoromethoxy and the combination of R¹ and R^{a} corresponds for each compound to one row of Table A

### Table 39

Compounds of the formula IB.2, in which R³ and R⁵ are methyl and the combination of R¹ and R^{a} corresponds for each compound to one row of Table A

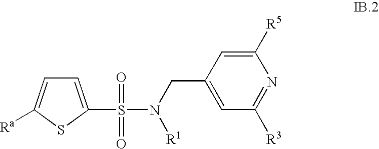

### Table 40

Compounds of the formula IB.2, in which R³ and R⁵ are fluorine and the combination of R¹ and R^{a} corresponds for each compound to one row of Table A

### Table 41

Compounds of the formula IB.2, in which R³ and R⁵ are chlorine and the combination of R¹ and R^{a} corresponds for each compound to one row of Table A

### Table 42

Compounds of the formula IB.2, in which R³ and R⁵ are methoxy and the combination of R¹ and R^{a} corresponds for each compound to one row of Table A

### Table 43

Compounds of the formula IB.2, in which R³ and R⁵ are trifluoromethoxy and the combination of R¹ and R^{a} corresponds for each compound to one row of Table A

### Table 44

Compounds of the formula IB.3, in which R² and R⁴ are methyl and the combination of R¹ and R^{a} corresponds for each compound to one row of Table A

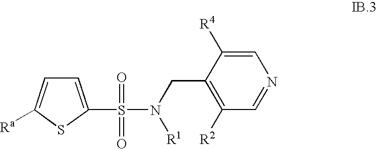

### Table 45

Compounds of the formula IB.3, in which R² and R⁴ are fluorine and the combination of R¹ and R^{a} corresponds for each compound to one row of Table A

### Table 46

Compounds of the formula IB.3, in which R² and R⁴ are chlorine and the combination of R¹ and R^{a} corresponds for each compound to one row of Table A

### Table 47

Compounds of the formula IB.3, in which R² and R⁴ are methoxy and the combination of R¹ and R^{a} corresponds for each compound to one row of Table A

### Table 48

Compounds of the formula IB.3, in which R² and R⁴ are trifluoromethoxy and the combination of R¹ and R^{a} corresponds for each compound to one row of Table A

### Table 49

Compounds of the formula IB.4′, in which R^{2a} and R^{3a} are hydrogen and the combination of R¹ and R^{a} corresponds for each compound to one row of Table A

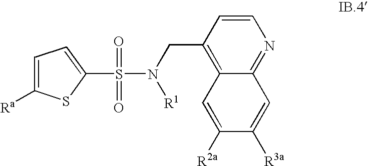

### Table 50

Compounds of the formula IB.4′, in which R^{2a} and R^{3a} are methyl and the combination of R¹ and R^{a} corresponds for each compound to one row of Table A

### Table 51

Compounds of the formula IB.4′, in which R^{2a} and R^{3a} are fluorine and the combination of R¹ and R^{a} corresponds for each compound to one row of Table A

### Table 52

Compounds of the formula IB.4′, in which R^{2a} and R^{3a} are chlorine and the combination of R¹ and R^{a} corresponds for each compound to one row of Table A

### Table 53

Compounds of the formula IB.4′, in which R^{2a} and R^{3a} are methoxy and the combination of R¹ and R^{a} corresponds for each compound to one row of Table A

### Table 54

Compounds of the formula IB.4′, in which R^{2a} and R^{3a} are trifluoromethoxy and the combination of R¹ and R^{a} corresponds for each compound to one row of Table A

### Table 55

Compounds of the formula IB.5, in which R² is methyl and the combination of R¹ and R^{a} corresponds for each compound to one row of Table A

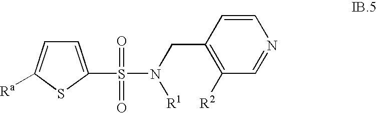

### Table 56

Compounds of the formula IB.5, in which R² is fluorine and the combination of R¹ and R^{a} corresponds for each compound to one row of Table A

### Table 57

Compounds of the formula IB.5, in which R² is chlorine and the combination of R¹ and R^{a} corresponds for each compound to one row of Table A

### Table 58

Compounds of the formula IB.5, in which R² is methoxy and the combination of R¹ and R^{a} corresponds for each compound to one row of Table A

### Table 59

Compounds of the formula IB.5, in which R² is trifluoromethoxy and the combination of R¹ and R^{a} corresponds for each compound to one row of Table A

### Table 60

Compounds of the formula IB.6, in which R³ is methyl and the combination of R¹ and R^{a} corresponds for each compound to one row of Table A

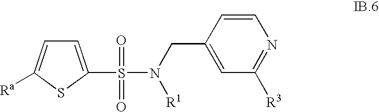

### Table 61

Compounds of the formula IB.6, in which R³ is fluorine and the combination of R¹ and R^{a} corresponds for each compound to one row of Table A

### Table 62

Compounds of the formula IB.6, in which R³ is chlorine and the combination of R¹ and R^{a} corresponds for each compound to one row of Table A

### Table 63

Compounds of the formula IB.6, in which R³ is methoxy and the combination of R¹ and R^{a} corresponds for each compound to one row of Table A

### Table 64

Compounds of the formula IB.6, in which R³ is trifluoromethoxy and the combination of R¹ and R^{a} corresponds for each compound to one row of Table A

TABLE A

| No. | R¹ | R^{a} |
|---|---|---|
| A-1 | H | C(CH₃)═NOCH₃ |
| A-2 | CH₃ | C(CH₃)═NOCH₃ |
| A-3 | H | C(CH₃)═NOCH₂CH₃ |
| A-4 | CH₃ | C(CH₃)═NOCH₂CH₃ |
| A-5 | H | C(CH₃)═NOCH₂CH═CH₂ |
| A-6 | CH₃ | C(CH₃)═NOCH₂CH═CH₂ |
| A-7 | H | C(CH₃)═NOCH₂C≡CH |
| A-8 | CH₃ | C(CH₃)═NOCH₂C≡CH |
| A-9 | H | C(CH₃)═NOCH₂CCl═CH₂ |
| A-10 | CH₃ | C(CH₃)═NOCH₂CCl═CH₂ |
| A-11 | H | H |
| A-12 | CH₃ | H |
| A-13 | H | CH₃ |
| A-14 | CH₃ | CH₃ |
| A-15 | H | CH₂CH₃ |
| A-16 | CH₃ | CH₂CH₃ |
| A-17 | H | CH₂CH₂CH₃ |
| A-18 | CH₃ | CH₂CH₂CH₃ |
| A-19 | H | CH(CH₃)₂ |
| A-20 | CH₃ | CH(CH₃)₂ |
| A-21 | H | CH₂CH₂CH₂CH₃ |
| A-22 | CH₃ | CH₂CH₂CH₂CH₃ |
| A-23 | H | CH(CH₃)CH₂CH₃ |
| A-24 | CH₃ | CH(CH₃)CH₂CH₃ |
| A-25 | H | CH₂CH(CH₃)₂ |
| A-26 | CH₃ | CH₂CH(CH₃)₂ |
| A-27 | H | C(CH₃)₃ |
| A-28 | CH₃ | C(CH₃)₃ |
| A-29 | H | OCH₃ |
| A-30 | CH₃ | OCH₃ |
| A-31 | H | OCH₂CH₃ |
| A-32 | CH₃ | OCH₂CH₃ |
| A-33 | H | OCH₂CH₂CH₃ |
| A-34 | CH₃ | OCH₂CH₂CH₃ |
| A-35 | H | OCH(CH₃)₂ |
| A-36 | CH₃ | OCH(CH₃)₂ |
| A-37 | H | OCH₂CH₂CH₂CH₃ |
| A-38 | CH₃ | OCH₂CH₂CH₂CH₃ |
| A-39 | H | OCH(CH₃)CH₂CH₃ |
| A-40 | CH₃ | OCH(CH₃)CH₂CH₃ |
| A-41 | H | OCH₂CH(CH₃)₂ |
| A-42 | CH₃ | OCH₂CH(CH₃)₂ |
| A-43 | H | OC(CH₃)₃ |
| A-44 | CH₃ | OC(CH₃)₃ |
| A-45 | H | CCl₃ |
| A-46 | CH₃ | CCl₃ |
| A-47 | H | CHF₂ |
| A-48 | CH₃ | CHF₂ |
| A-49 | H | CF₃ |
| A-50 | CH₃ | CF₃ |
| A-51 | H | CHClF |
| A-52 | CH₃ | CHClF |
| A-53 | H | CH₂CHF₂ |
| A-54 | CH₃ | CH₂CHF₂ |
| A-55 | H | CH₂CF₃ |
| A-56 | CH₃ | CH₂CF₃ |
| A-57 | H | CF₂CF₃ |
| A-58 | CH₃ | CF₂CF₃ |
| A-59 | H | OCHCl₂ |
| A-60 | CH₃ | OCHCl₂ |
| A-61 | H | OCCl₃ |
| A-62 | CH₃ | OCCl₃ |
| A-63 | H | OCH₂F |
| A-64 | CH₃ | OCH₂F |
| A-65 | H | OCHF₂ |
| A-66 | CH₃ | OCHF₂ |
| A-67 | H | OCF₃ |
| A-68 | CH₃ | OCF₃ |
| A-69 | H | OCH₂CHF₂ |
| A-70 | CH₃ | OCH₂CHF₂ |
| A-71 | H | OCH₂CF₃ |
| A-72 | CH₃ | OCH₂CF₃ |
| A-73 | H | OCH₂CHClF |
| A-74 | CH₃ | OCH₂CHClF |
| A-75 | H | OCH₂CCl₃ |
| A-76 | CH₃ | OCH₂CCl₃ |
| A-77 | H | OCF₂CF₃ |
| A-78 | CH₃ | OCF₂CF₃ |
| A-79 | H | C₆H₅ |
| A-80 | CH₃ | C₆H₅ |
| A-81 | H | OC₆H₅ |
| A-82 | CH₃ | OC₆H₅ |
| A-83 | H | 4-[(CH₃)₃C]—C₆H₄ |
| A-84 | CH₃ | 4-[(CH₃)₃C]—C₆H₄ |
| A-85 | H | Br |
| A-86 | CH₃ | Br |
| A-87 | H | Cl |
| A-88 | CH₃ | Cl |
| A-89 | H | 4-C₆H₅—C₆H₄ |
| A-90 | CH₃ | 4-C₆H₅—C₆H₄ |
| A-91 | H | 4-Cl—C₆H₄ |
| A-92 | CH₃ | 4-Cl—C₆H₄ |
| A-93 | H | 4-(F₃C—O)—C₆H₄ |
| A-94 | CH₃ | 4-(F₃C—O)—C₆H₄ |
| A-95 | H | 4-(H₃C—O)—C₆H₄ |
| A-96 | CH₃ | 4-(H₃C—O)—C₆H₄ |
| A-97 | H | 4-(F₃C)—C₆H₄ |
| A-98 | CH₃ | 4-(F₃C)—C₆H₄ |
| A-99 | H | 4-(H₃C)—C₆H₄ |
| A-100 | CH₃ | 4-(H₃C)—C₆H₄ |
| A-101 | H | 4-(H₅C₂)—C₆H₄ |
| A-102 | CH₃ | 4-(H₅C₂)—C₆H₄ |
| A-103 | H | 4-(n-H₇C₃)—C₆H₄ |
| A-104 | CH₃ | 4-(n-H₇C₃)—C₆H₄ |
| A-105 | H | 4-[(H₃C)₂CH]—C₆H₄ |
| A-106 | CH₃ | 4-[(H₃C)₂CH]—C₆H₄ |

The compounds according to the invention can be obtained by different routes, e.g as cited in WO 05/033081 und literature cited therein.

The compounds of the formula IB can be prepared by reacting compounds II with thienylsulfonyl halides III, wherein the variables have the meaning as defined above for compounds of formula I and L is halogen, preferably chlorine.

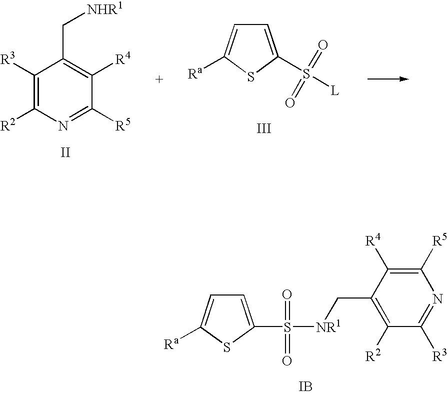

The starting materials are generally reacted with one another in equimolar amounts. In terms of yield, it may be advantageous to employ an excess of II, based on III.

Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, dioxane, anisole and tetrahydrofuran and dimethoxyethane, ketones, such as acetone, methyl ethyl ketone, diethyl ketone and tert.-butyl methyl ketone, nitriles, such as acetonitrile, and also dimethyl sulfoxide, dimethylformamide and dimethylacetamide, particularly preferably ethers, such as tetrahydrofuran, dioxane and dimethoxyethane. It is also possible to use mixtures of the solvents mentioned.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to bases such as sodium carbonate, potassium carbonate, cesium carbonate, triethylamine and sodium bicarbonate.

The bases are generally employed in equimolar amounts; however, they can also be employed in excess or, if appropriate, as solvent.

Compounds II can be prepared for example by reduction of the corresponding nitrile oxime or amide. Appropriate methods and for the synthesis of the corresponding starting materials are know to those skilled in the art or can be found in J. Org. Chem. 23 714 1958; J. Prakt. Chem. 336 (8) 695, 1994; Chem Pharm Bull 1973 21 1927, U.S. Pat. No. 4,439,609; Houben-Weyl Band 10/4 Thieme Stuttgart, 1968, Band 11/2 1957, Band E5 1985, Heterocyclic compounds Vol 14 Part 1-4 Wiley N.Y. 1974-1975; Methods in Science of Synthesis, Volume 15; Tetrahedron 57, 2001, p. 4489; Eur. J. Org. Chem, 2001, p. 1371; Tetrahedron 57, 2001, p. 4059, US 2005 0239791, Heterocycles 65, 8, p 2005; European Journal of Organic Chemistry, 2003, 8, pp. 1559.

Compounds of the formulae IB.1, IB.2, IB.3, IB.5 and IB.6 wherein R^{a} is unsubstituted or substituted phenyl as defined herein for these compounds can also be obtained by a Suzuki coupling of the respective compounds wherein R^{a} is halogen, preferably bromine or iodine, by coupling with a boronic acid R^{a}—B(OH)₂ wherein R^{a} is unsubstituted or substituted phenyl as defined herein for compounds of the formulae IB.1, IB.2, IB.3, IB.5 and IB.6.

This Suzuki coupling is usually carried out at temperatures of from 20° C. to 180° C., preferably from 40° C. to 120° C., in an inert organic solvent in the presence of a base and a platinum metal, in particular a palladium catalyst (literature see e.g. Synth. Commun. Vol. 11, p. 513 (1981); Acc. Chem. Res. Vol. 15, pp. 178-184 (1982); Chem. Rev. Vol. 95, pp. 2457-2483 (1995); Organic Letters Vol. 6 (16), p. 2808 (2004)).

Suitable catalysts are in particular tetrakis(triphenylphosphine)platinum(0); tetra-kis(triphenylphosphine)palladium(0); bis(triphenylphosphine)palladium(II) chloride; bis(acetonitrile)palladium(II) chloride; [1,1′-bis(diphenylphosphino)ferrocene]-palladium(II) chloride/methylene chloride(1:1) complex; bis[bis-(1,2-diphenylphosphino)ethane]palladium(0); bis(bis-(1,2-diphenylphosphino)butane]-palladium(II) chloride; palladium(II) acetate; palladium(II) chloride; and palladium(II) acetate/tri-o-tolylphosphine complex, it is also possible to use a polymer bound phosphine-Pd-complex, eg. polystyryl-triphenylphosphin-Pd.

The starting materials are generally reacted with one another in equimolar amounts. In terms of yield, it may be advantageous to use an excess of boronic acid.

The boronic acids are commercial available or can be synthesized according to methods known to those skilled in the art, e.g. as in WO 02/042275.

The quinoline compounds of the formula I.4 wherein X is phenyl can be prepared in a similar manner as outlined above by reacting quinolineamine II.1 wherein R² and R³ together with the carbon atom to which they are attached form a phenyl ring and the other variables are as defined for compounds of formula IA.4 with a halophenylsulfonylchloride. Compounds of formula IA.4′ wherein R^{a} is unsubstituted or substituted phenyl as defined herein for compounds IA.4′ can be prepared by a Suzuki coupling of the corresponding compounds I.4 wherein X is phenyl and R^{a} is halogen and in the 4-position with a boronic acid IV wherein R^{b} is as defined in compounds IA.4′.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I by customary modifications of the synthesis routes described.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

The compounds of the general formula I according to the invention show high activity against harmful arthropodes. They may act by contact or may be stomach-acting, or have systemic or residual action. Contact action means that the pest is killed by coming into contact with a compound I or with material that releases compound I. Stomach-acting means that the pest is killed if it ingests a pesticidially effective amount of the compound I or material containing a pesticidally effective amount of compound I. Systemic action means that the compound is absorbed into the plant tissues of treated plant and the pest is controlled, if it eats plant tissue or sucks plant-sap. Compounds I are in particular suitable for controlling the following pests:

insects from the order of Lepidoptera, for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalls, Cirphis unipuncta, Cydia pomonella, Dendrolimus pini Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera eridania, Spodoptera frugiperda, Spodoptera littoralis, Spodoptera litura, Thaumatopoea pityocampa, Tortrix viridana, Trichoplusia ni and Zeiraphera canadensis,

from the order of Coleoptera (beetles), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

from the order of Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

from the order of Thysanoptera (thrips), e.g. Dichromothrips spp., Frankliniella fusca, Frankliniella occidentalis, Frankliniella tritici, Scirtothrips citri, Thrips oryzae, Thrips palmi and Thrips tabaci,

ants, bees, wasps, sawflies (Hymenoptera) e.g. Athalia rosae, Atta cephalotes, Atta cephalotes, Atta laevigata, Atta robusta, Atta capiguara, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

from the order of Isoptera(termites), e.g. Calotermes flavicollis, Heterotermes aureus, Leucotermes flavipes, Reticulitermes flavipes, Reticulitermes virginicus, Reticulitermes lucifugus, a Termes natalensis, and Coptotermes formosanus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypi, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus,

crickets, grasshoppers, locusts (Orthoptera), e.g. Acheta domestics, Blatta orientalis, Blattella germanica, Calliptamus italicus, Chortoicetes terminifera, Dociostaurus maroccanus, Forficula auricularia, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Periplaneta americana, Schistocerca americana, Schistocerca peregrina, Schistocerca gregaria, Stauronotus maroccanus, Tachycines asynamorus, Tachycines asynamorus, Zonozerus variegatus,

Arachnoidea, such as Acarina, e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus hermsi, Ornithodorus turicata, Ornithonyssus bacoti, Ornithodorus moubata, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis and Oligonychus pratensis; Araneida, e.g. Latrodectus mactans, and Loxosceles reclusa,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

For use in a method according to the present invention, the compounds I can be converted into the customary formulations, e.g. solutions, emulsions, suspensions, dusts, powders, pastes and granules. The use form depends on the particular purpose; it is intended to ensure in each case a fine and uniform distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7515-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and binders.

Examples of suitable solvents water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are carriers such as ground natural minerals (e.g. kaolins, clays, talc, chalk) and ground synthetic minerals (e.g. highly disperse silica, silicates);

Suitable emulsifiers are nonionic and anionic emulsifiers (e.g. polyoxyethylene fatty alcohol ethers, alkylsulfonates and arylsulfonates);

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Also anti-freezing agents such as glycerin, ethylene glycol, propylene glycol and bactericides such as can be added to the formulation.

Suitable antifoaming agents are for example antifoaming agents based on silicon or magnesium stearate.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100% by weight, preferably 95% to 100% by weight (according to NMR spectrum).

The following are examples of formulations:

1. Products for Dilution with Water

### A Soluble Concentrates (SL, LS)

10 parts by weight of a compound according to the invention are dissolved in water or in a water-soluble solvent. As an alternative, wetters or other auxiliaries are added. The active ingredient dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

### D Emulsions (EW, EO, ES)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

### E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

### F Water-Dispersible Granules and Water-Soluble Granules (WG, SG, SS, WS)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

### G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active ingredient.

### 2. Products to be Applied Undiluted

### H Dustable Powders (DP, DS)

5 parts by weight of a compound according to the invention are ground finely and mixed intimately with 95% of finely divided kaolin. This gives a dustable product.

### I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

### J ULV Solutions (UL, LS)

10 parts by weight of a compound according to the invention are dissolved in an organic solvent, for example xylene. This gives a product to be applied undiluted.

The active ingredients can be used as such, in the form of their formulations or the use forms prepared therefrom, eg. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

The compounds of formula I are also suitable for the protection of the seed, plant propagules and the seedlings' roots and shoots, preferably the seeds, against soil pests and also for the treatment plant seeds which tolerate the action of herbicides or fungicides or insecticides owing to breeding, including genetic engineering methods.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds.

The seed treatment application of the compounds of formula I or formulations containing them is carried out by spraying or dusting the seeds before sowing of the plants and before emergence of the plants.

The invention also relates to the propagation product of plants, and especially the treated seed comprising, that is, coated with and/or containing, a compound of formula I or a composition comprising it. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed comprises the inventive compounds or compositions comprising them in an amount of from 0.1 g to 10 kg per 100 kg of seed.

Preferred FS formulations of compounds of formula I for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®), Polymin®), polyethers and copolymers derived from these polymers.

Compositions of this invention may also contain other active ingredients, for example other pesticides such as insecticides, fungicides, biocides and herbicides, fertilizers such as ammonium nitrate, urea, potash, and superphosphate, phytotoxicants and plant growth regulators, oils, wetters, adjuvants, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents usually are admixed with the agents according to the invention in a weight ratio of 1:100 to 100:1.

The following list of pesticides together with which the compounds according to the invention can be used, is intended to illustrate the possible combinations, but not to impose any limitation:

A.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
A.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
A.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
A.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
A.5. Nicotinic receptor agonists/antagonists compounds: clothianidin, dinotefuran, imidacloprid, thiamethoxam, nitenpyram, acetamiprid, thiacloprid;
the thiazol compound of formula (Γ¹)

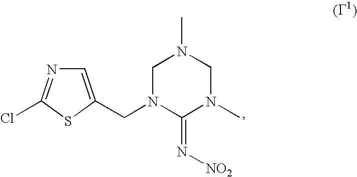

A.6. GABA antagonist compounds: acetoprole, endosulfan, ethiprole, fipronil, vaniliprole, pyrafluprole, pyriprole, the phenylpyrazole compound of formula Γ²

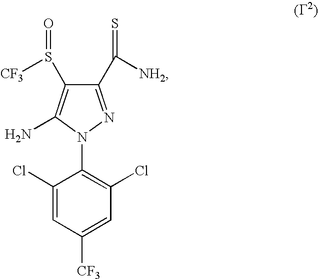

A.7. Macrocyclic lactone insecticides: abamectin, emamectin, milbemectin, lepimectin, spinosad;
A.8. METI I compounds: fenazaquin, pyridaben, tebufenpyrad, tolfenpyrad, flufenerim;
A.9. METI II and III compounds: acequinocyl, fluacyprim, hydramethylnon;
A.10. Uncoupler compounds: chlorfenapyr;
A.11. Oxidative phosphorylation inhibitor compounds: cyhexatin, diafenthiuron, fenbutatin oxide, propargite;
A.12. Moulting disruptor compounds: cyromazine;
A.13. Mixed Function Oxidase inhibitor compounds: piperonyl butoxide;
A.14. Sodium channel blocker compounds: indoxacarb, metaflumizone,
A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet, N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, anthranilamide compounds of formula Γ³

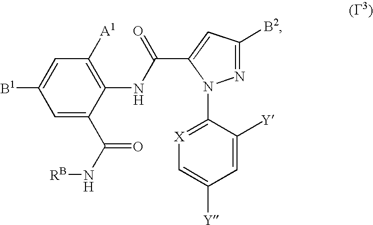

wherein. A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂, and malononitrile compounds as described in JP 2002 284608, WO 02/89579, WO 02/90320, WO 02/90321, WO 04/06677, WO 04/20399, or JP 2004 99597.

The aforementioned compositions are particularly useful for protecting plants against infestation of said pests or to combat these pests in infested plants.

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cock-roaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethyl-formamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

The compounds of formula I and its compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of compounds of formula I or with a salt thereof or with a composition, containing a pesticidally effective amount of a compound of formula I or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

Compounds of formula I and compositions comprising them can also be used for controlling and preventing infestations and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I and compositions comprising them are suitable for systemic and/or non-systemic control of ecto- and/or endoparasites. They are active against all or some stages of development.

Administration can be carried out both prophylactically and therapeutically. Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably I ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Generally it is favorable to apply solid formulations which release compounds of formula I in total amounts of 10 mg/kg to 300 mg/kg, preferably 20 mg/kg to 200 mg/kg. The active compounds can also be used as a mixture with synergists or with other active compounds which act against pathogenic endo- and ectoparasites.

In general, the compounds of formula I are applied in parasiticidally effective amount—meaning the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The present invention is now illustrated in further detail by the following examples.

### I. Synthesis of Compounds I:

The procedures described in the synthesis examples below were used to prepare further compounds I by appropriate modification of the starting compounds. The compounds thus obtained are listed in the tables below, together with physical data.

### PREPARATION EXAMPLE 1

### Preparation of 4-acetyl-N-pyridin-4-ylmethylphenylsulfonamide

At −10° C., a solution of 4.95 g (45.7 mmol) of 4-(aminomethyl)pyridine (4-picolylamine) in 10 ml of diethyl ether was added dropwise to a solution of 10 g (45.7 mmol) of 4-acetylsulfonyl chloride in 150 ml of diethyl ether, and the solution was then stirred at 20-25° C. for about 18 hours. The product was filtered off with suction and the residue was washed with dilute NaHCO₃ solution and water and then dried. This gave 5.2 g of the title compound of m.p.: 162-167° C.

### PREPARATION EXAMPLE 2

### Preparation of 4-(1-ethoxyiminoethyl)-N-pyridin-4-ylmethylphenylsulfonamide

0.42 g of a 40% strength aqueous O-ethylhydroxylamine solution was added to a solution of 0.4 g (1.3 mmol) of the compound from Example I in 20 ml of methanol. Using 10% strength hydrochloric acid, the mixture was acidified to pH 4, and the solution was then stirred at 20-25° C. for about 18 hours. The reaction solution was poured into water and adjusted to pH 8 using NaHCO₃. The mixture was then extracted with methyl tert-butyl ether (MtBE), and the combined organic phases were washed with water and dried. Removal of the solvent gave 0.4 g of the title compound as a viscous oil.

¹H-NMR (δ, CDCl₃,): 8.5 (d, 2H); 7.5 (m, 4H); 7.1 (d, 2H); 5.0 (t, 1H); 4.25 (q, 2H); 4.1 (d, 2H); 2.25 (s, 3H); 1.3 (t, 3H).

### PREPARATION EXAMPLE 3

### Preparation of 4-(1-ethoxyimino-ethyl)-N-methyl-N-pyridin-4-ylmethyl-phenylsulfonamide

0.4 g (1.2 mmol) of the compound from Example 2 was added to a slurry of 0.04 g (1.32 mmol) of NaH (95% pure) in 50 ml of dimethylformamide (DMF), and the mixture was then stirred at 20-25° C. for 10 min. A solution of 0.17 g (1.2 mmol) of iodomethane in 10 ml of DMF was then added dropwise, and the combined reaction solution was stirred at 20-25° C. for about 18 hours, poured into water and then extracted with MtBE. The organic phases were washed with water and then dried. Removal of the solvent gave 0.3 g of the title compound as a viscous oil.

¹H-NMR (δ, CDCl₃,): 8.6 (d, 2H); 7.8 (m, 4H); 7.25 (d, 2H); 4.25 (q, 2H); 4.1 (d, 2H); 2.6 (s, 3H); 2.25 (s, 3H); 1.25 (t, 3H).

### PREPARATION EXAMPLE 4

### Preparation of 5-bromo-thiophene-2-sulfonic acid (quinoline-4-ylmethyl)-amide

5 g (31.6 mmol) quinoline-4-methyleneamine (commercial compound) and 3.5 g (34.8 mmol) triethylamine in 40 ml of methylenehloride was treated with 8.52 g (31.6 mmol) of 5-bromothienyl-2-sulfonic acid chloride. After 48 hours, 200 ml water were added and the precipitate was collected and subjected to column chromatography (cyclohexane/ethyl acetate 7/3) to yield 2.8 g of the title compound.

1H-NMR (δ, d⁶-DMSO): 8.8 (m, 1H); 8.7 (s, 1H); 8.3-8.0 (m, 2H), 7.7 (m, 1H), 7.7 (m, 1H), 7.5 (m, 1H), 7.3 (m, 1H), 4.6 (s, 2H).

### PREPARATION EXAMPLE 5

### Preparation of 5-(4-chlorophenyl)-thiophene-2-sulfonic acid (quinoline-4-ylmethyl)-amide

0.3 g (0.8 mmol) of 5-bromo-thiophene-2-sulfonic acid (quinoline-4-ylmethyl)-amide, 0.278 g (1.6 mmol) 4-chlorophenylboronic acid, 1.6 ml of a 2.6 mol aqueous solution of K₂CO₃ and 0.8 g (0.7 mmol) of polystryene-triphenylphosphine-Pd (Argonaut) in 15 ml of tetrahydrofuran were heated to 75° C. for 48 hours. After filtration and washing with 25 ml of tetrahydrofuran the volatiles were removed in vacuo and the crude product was subjected to chromatography (cyclohexane/ethylacetate 7/3) to afford 0.17 g of the title product, mp. 185-186° C., MS: m/e [M+H⁺]=414.9.

TABLE I

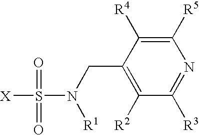

| | | | | | | | phys. data (mp. [° C.]; |
|---|---|---|---|---|---|---|---|
| | | | | | | | ¹H-NMR δ [ppm]; |
| No. | X | R¹ | R² | R³ | R⁴ | R⁵ | MS m/e [M + H⁺]) |
| I-1 | 4-(C[CH₃]═NOCH₃)—C₆H₄ | H | H | H | H | H | 4.2 (2H); 4.0 (3H); |
| | | | | | | | 2.25 (3H) |
| I-2 | 4-(C[CH₃]═NOCH₂CH₃)—C₆H₄ | H | H | H | H | H | 4.25 (2H); 4.2 (2H); |
| | | | | | | | 2.25 (3H); 1.3 (3H) |
| I-3 | 4-(C[CH₃]═NOCH₂CH═CH₂)—C₆H₄ | H | H | H | H | H | 5.2 (2H); 4.7 (2H); |
| | | | | | | | 4.05 (2H); 2.25 (3H) |
| I-4 | 4-(C[CH₃]═NOCH(CH₃)₂)—C₆H₄ | H | H | H | H | H | 4.45 (1H); 4.2 (2H); |
| | | | | | | | 2.25 (3H); 1.3 (6H) |
| I-5 | 4-(C[CH₃]═NOCH₂C≡CH)—C₆H₄ | H | H | H | H | H | 4.8 (2H); 4.2 (2H); |
| | | | | | | | 2.5 (1H); 2.3 (3H) |
| I-6 | 4-(C[CH₃]═NO(CH₂)₅CH₃)—C₆H₄ | H | H | H | H | H | 4.2 (2H); 4.1 (2H); |
| | | | | | | | 2.25 (3H); 0.9 (3H) |
| I-7 | 4-(C[CH₃]═NOCH₂C₆H₅)—C₆H₄ | H | H | H | H | H | 5.3 (2H); 4.25 (2H); |
| | | | | | | | 2.25 (3H) |
| I-8 | 4-(C[CH₃]═NOCH₃)—C₆H₄ | CH₃ | H | H | H | H | 4.2 (2H); 4.05 (3H); |
| | | | | | | | 2.65 (3H); 2.25 (3H) |
| I-9 | 4-(C[CH₃]═NOCH₂CH₃)—C₆H₄ | CH₃ | H | H | H | H | 4.25 (2H); 4.2 (2H); |
| | | | | | | | 2.7 (3H); 1.35 (3H) |
| I-10 | 4-(C[CH₃]═NOCH₂CH₃)—C₆H₄ | CH₂CH₃ | H | H | H | H | 4.3 (2H); 4.2 (2H); |
| | | | | | | | 3.2 (2H); 2.2 (3H); |
| | | | | | | | 1.3 (3H); 0.9 (3H) |
| I-11 | 4-(C[CH₃]═NOCH₂CH₃)—C₆H₄ | CH₂C≡CH | H | H | H | H | 4.35 (2H); 4.25 (2H); |
| | | | | | | | 4.0 (2H); 2.25 (1H) |
| I-12 | 4-(C[CH₃]═NOCH₂CH₃)—C₆H₄ | C₆H₅CH₂ | H | H | H | H | 4.3 (2H); 4.25 (2H); |
| | | | | | | | 4.2 (2H); 2.25 (3H) |
| I-13 | 4-(C[CH₃]═NOCH₂CH₃)—C₆H₄ | CH₂CH═CH₂ | H | H | H | H | 5.5 (1H); 5.0 (2H); |
| | | | | | | | 4.3 (2H); 3.75 (2H) |
| I-14 | 4-CH₃O—OC—C₆H₄ | H | —CH═CH—CH═CH— | | H | H | 8.45 (1H); 8.0 (1H); |
| | | | | | | | 7.4 (1H); 4.5 (2H) |
| I-15 | 4-CH₃—C₆H₄ | H | —CH═CH—CH═CH— | | H | H | 8.1 (1H); 7.3 (1H); |
| | | | | | | | 4.65 (2H); 1.3 (9H) |
| I-16 | 4-CH₃—O—C₆H₄ | H | —CH═CH—CH═CH— | | H | H | 8.1 (1H); 4.6 (1H); |
| | | | | | | | 3.0 (1H); 1.3 (6H) |
| I-17 | 4-Cl—C₆H₄ | H | —CH═CH—CH═CH— | | H | H | m/e 333 |
| I-18 | 2.5-Cl₂—C₆H₃ | H | —CH═CH—CH═CH— | | H | H | m/e 367 |
| I-19 | 1-naphthyl | H | —CH═CH—CH═CH— | | H | H | m/e 349 |
| I-20 | 2-CH₃-6-CF₃—C₆H₃ | H | —CH═CH—CH═CH— | | H | H | m/e 381 |
| I-21 | 2-Cl-5-CH₃O—C₆H₃ | H | —CH═CH—CH═CH— | | H | H | m/e 363 |
| I-22 | 2.4-Cl₂—C₆H₃ | H | —CH═CH—CH═CH— | | H | H | m/e 367 |
| I-23 | 4-CN—C₆H₄ | H | —CH═CH—CH═CH— | | H | H | m/e 324 |
| I-24 | 2.6-Cl₂—C₆H₃ | H | —CH═CH—CH═CH— | | H | H | m/e 367 |
| I-25 | 2-Br—C₆H₄ | H | —CH═CH—CH═CH— | | H | H | m/e 379 |
| I-26 | 2.3-Cl₂—C₆H₃ | H | —CH═CH—CH═CH— | | H | H | m/e 367 |
| I-27 | 3.4-(CH₃O)₂—C₆H₃ | H | —CH═CH—CH═CH— | | H | H | m/e 358 |
| I-28 | 2-CH₃-6-Cl—C₆H₃ | H | —CH═CH—CH═CH— | | H | H | m/e 347 |
| I-29 | 2-Cl—C₆H₄ | H | —CH═CH—CH═CH— | | H | H | m/e 333 |
| I-30 | C₆H₅ | H | —CH═CH—CH═CH— | | H | H | 8.45 (1H); 8.0 (1H); |
| | | | | | | | 7.4 (1H); 4.5 (2H) |
| I-31 | 4-(CH₃)₃C—C₆H₄ | H | —CH═CH—CH═CH— | | H | H | 8.1 (1H); 7.3 (1H); |
| | | | | | | | 4.65 (2H); 1.3 (9H) |
| I-32 | 4-(CH₃)₂CH—C₆H₄ | H | —CH═CH—CH═CH— | | H | H | 8.1 (1H); 4.6 (1H); |
| | | | | | | | 3.0 (1H); 1.3 (6H) |
| I-33 | 4-CF₃O—C₆H₄ | H | —CH═CH—CH═CH— | | H | H | 8.75 (1H); 8.0 (1H); |
| | | | | | | | 5.8 (1H); 4.7 (2H) |
| I-34 | 4-CHF₂CF₂O—C₆H₄ | H | —CH═CH—CH═CH— | | H | H | 8.8 (1H); 4.7 (2H) |
| I-35 | 4-CH₃(O)C—C₆H₄ | H | —CH═CH—CH═CH— | | H | H | 8.8 (1H); 7.3 (1H); |
| | | | | | | | 4.6 (2H); 2.6 (3H) |
| I-36 | 4-(C[CH₃]═NOCH₃)—C₆H₄ | H | —CH═CH—CH═CH— | | H | H | 5.2 (NH); 4.6 (2H); |
| | | | | | | | 4.0 (3H); 2.2 (3H) |
| I-37 | 4-(C[CH₃]═NOCH₂CH₃)—C₆H₄ | H | —CH═CH—CH═CH— | | H | H | 5.0 (NH); 4.6 (2H); |
| | | | | | | | 4.3 (3H); 2.3 (3H); |
| | | | | | | | 1.35 (3H) |
| I-38 | 4-([CH₃]═NOCH(CH₃)₂)—C₆H₄ | H | —CH═CH—CH═CH— | | H | H | 5.1 (NH); 4.6 (2H); |
| | | | | | | | 4.5 (3H); 2.25 (3H); |
| | | | | | | | 1.3 (6H) |
| I-39 | 4-(C[CH₃]═NOCH₂CH═CH₂)—C₆H₄ | H | —CH═CH—CH═CH— | | H | H | 5.3 (NH); 5.2 (2H); |
| | | | | | | | 4.75 (2H); 4.6 (2H); |
| | | | | | | | 2.35 (3H) |
| I-40 | 4-(C[CH₃]═NOCH₂C≡CH)—C₆H₄ | H | —CH═CH—CH═CH— | | H | H | 5.1 (NH); 4.8 (2H); |
| | | | | | | | 4.6 (2H); 2.5 (2H); |
| | | | | | | | 2.3 (3H) |
| I-41 | 4-(C[CH₃]═NO(CH₂)₅CH₃)—C₆H₄ | H | —CH═CH—CH═CH— | | H | H | 5.2 (NH); 4.6 (2H); |
| | | | | | | | 4.2 (2H); 2.25 (2H); |
| | | | | | | | 0.9 (3H) |
| I-42 | 4-(C[CH₃]═NOCH₂C₆H₅)—C₆H₄ | H | —CH═CH—CH═CH— | | H | H | 5.3 (2H); 5.2 (NH); |
| | | | | | | | 4.8 (2H); 2.3 (3H) |
| I-43 | C₆H₅ | CH₃ | —CH═CH—CH═CH— | | H | H | 4.6 (2H); 2.7 (3H) |
| I-44 | 5-Cl-thiophen-2-yl | H | H | H | H | H | m/e 339 |
| I-45 | thiophen-2-yl | H | H | H | H | H | m/e 305 |
| I-46 | 5-([4-C(CH₃]₃]—C₆H₄)-thiophen-2-yl | H | H | H | H | H | 166 |
| I-47 | 5-Br-thiophen-2-yl | H | H | H | H | H | 148 |
| I-48 | 4-NO₂-5-Cl-thiophen-2-yl | H | H | H | H | H | 182 |
| I-49 | 5-Cl-1,3-(CH₃)₂-1H-pyrazol-4-yl | H | H | H | H | H | 147 |
| I-50 | 3-Br-2,5-Cl₂-thiophen-4-yl | H | H | H | H | H | 106 |
| I-51 | 1-CH₃-1H-imidazol-4-yl | H | H | H | H | H | 125 |
| I-52 | 5-(4-tert.-butylphenyl)-thien-2-yl | H | H | H | H | H | 166 |
| I-53 | 5-chloro-1,3-dimethyl-1H-pyrazol-4-yl | H | H | H | H | H | 147 |
| I-54 | 5-bromothien-2-yl | H | H | H | H | H | 148 |
| I-55 | 5-chloro-4-nitro-thien-2-yl | H | H | H | H | H | 182 |
| I-56 | 1-methyl-1H-imidazol-4-yl | H | H | H | H | H | 125 |
| I-57 | 4-bromo-2,5-dichloro-thien-3-yl | H | H | H | H | H | 106 |
| I-58 | 5-biphenyl-44-yl-thien-2-yl | H | H | H | H | H | 204 |
| I-59 | 5-(4-trifluoromethoxyphenyl)-thien-2-yl | H | H | H | H | H | 138 |
| I-60 | 5-(4-Propyl-phenyl)-thiophen-2-yl | H | H | H | H | H | 144 |
| I-61 | 5-(4-ethyl-phenyl)-thien-2-yl | H | H | H | H | H | 144 |
| I-62 | 5-(3-trifluoromethyl-phenyl)-thien-yl | H | H | H | H | H | 130 |
| I-63 | 5-(4-chlorophenyl)-thien-2-yl | H | H | H | H | H | 176 |
| I-64 | 5-(4-trifluoromethyl-phenyl)-thien-yl | H | H | H | H | H | 165 |
| I-65 | 5-(4-methoxy-phenyl)-thien-2-yl | H | H | H | H | H | 175 |
| I-66 | 5-(4-trifluoromethyl-phenyl)-thien-2-yl | Propargyl | H | H | H | H | 116 |
| I-67 | 5-(4-trifluoromethyl-phenyl)-thien-2-yl | n-Propyl | H | H | H | H | 97 |
| I-68 | 5-(4-trifluoromethyl-phenyl)-thien-2-yl | Methyl | H | H | H | H | 101 |
| I-69 | 5-(4-isopropyl-phenyl)-thien-2-yl | H | H | H | H | H | 125 |

| | | | | | | |
|---|---|---|---|---|---|---|
| I-70 | 5-bromo-thien-2-yl | H | —CH═CH—CH═CH— | H | H | 137 |
| I-71 | 5-(4-trifluoromethyl-phenyl)-thien-2-yl | H | —CH═CH—CH═CH— | H | H | 180 |
| I-72 | 4-tert.butyl-phenyl | Methyl | —CH═CH—CH═CH— | H | H | 145-148 |
| I-73 | 5-(4-chloro-phenyl)-thien-2-yl | H | —CH═CH—CH═CH— | H | H | 185-187 |
| I-74 | 4-(4-methoxy-phenyl)-phenyl | H | —CH═CH—CH═CH— | H | H | 158-159 |

### II. Assessment of the Activity Against Animal Pest

1 Cotton Aphid (Aphis gossypii), Mixed Life Stages

The active compounds were formulated in 50:50 acetone:water and 100 ppm Kinetic® surfactant.

Cotton plants at the cotyledon stage were infested prior to treatment by placing a heavily infested leaf from the main aphid colony on top of each cotyledon. The aphids were allowed to transfer overnight and the host leaf was removed. The infested cotyledons were then dipped and agitated in the test solution for 3 seconds and allowed to dry in a fume hood. Test plants were maintained under fluorescent lighting in a 24-hr photoperiod at 25° C. and 20-40% relative humidity. Aphid mortality on the treated plants, relative to mortality on untreated check plants, was determined after 5 days.

In this test, the compounds I-36 and 1-71 at 300 ppm showed a mortality of at least 90% in comparison with untreated controls.

2. Southern Armyworm (Spodoptera eridania), 2^{nd}-3^{rd} Instar Larvae

The active compounds were formulated as a 10.000 ppm solution in a mixture of 35% acetone and water, which was diluted with water, if needed.

Sieva lima bean foliage, expanded to the first true leaves, were dipped and agitated in the test solution for 3 seconds and then allowed to dry in a fume hood. The treated plant was then placed in 25-cm plastic perforated zip enclosure bags, ten 2^{nd}-instar larvae were added, and the bags sealed. After 4 days, observations were made of mortality, plant feeding, and of any interference with larval growth.

In this test, the compounds I-36, I-66 and I-65 at 300 ppm showed a mortality of at least 90% in comparison with untreated controls.

3. Tobacco Budworm (Heliothis virescens)

Two-leaf cotton plants are utilized for bioassays. Excised plant leaves are dipped into 1:1 acetone/water dilutions of the active compounds. After the leaves have dried, they are individually placed onto water-moistened filter paper on the bottoms of Petri dishes. Each dish is infested with 5-7 larvae and covered with a lid. Each treatment dilution is replicated 4 times. Test dishes are held at approximately 27° C. and 60% humidity. Numbers of live and morbid larvae are assessed in each dish at 5 days after treatment application, and percent mortality is calculated.

4. Colorado Potato Beetle (Leptinotarsa decemlineata)

Potato plants are utilized for bioassays. Excised plant leaves are dipped into 1:1 acetone/water dilutions of the active compounds. After the leaves have dried, they are individually placed onto water-moistened filter paper on the bottoms of Petri dishes. Each dish is infested with 5-7 larvae and covered with a lid. Each treatment dilution is replicated 4 times. Test dishes are held at approximately 27° C. and 60% humidity. Numbers of live and morbid larvae are assessed in each dish at 5 days after treatment application, and percent mortality is calculated.

5. Green Peach Aphid (Myzus persicae)

The active compounds were formulated in 50:50 acetone:water and 100 ppm Kinetic™ surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, the compound I-74 at 300 ppm showed a mortality of at least 75% in comparison with untreated controls.

6. Silverleaf Whitefly (Bemisia argentifolii)

The active compounds are formulated in 50:50 acetone:water and 100 ppm Kinetic™ surfactant.

Selected cotton plants are grown to the cotyledon state (one plant per pot). The cotyledons are dipped into the test solution to provide complete coverage of the foliage and placed in a well-vented area to dry. Each pot with treated seedling is placed in a plastic cup and 10 to 12 whitefly adults (approximately 3-5 day old) are introduced. The insects are collected using an aspirator and an 0.6 cm, non-toxic Tygon tubing connected to a barrier pipette tip. The tip, containing the collected insects, is then gently inserted into the soil containing the treated plant, allowing insects to crawl out of the tip to reach the foliage for feeding. The cups are covered with a re-usable screened lid (150 micron mesh polyester screen PeCap from Tetko Inc). Test plants are maintained in the holding room at about 25° C. and 20-40% relative humidity for 3 days avoiding direct exposure to the fluorescent light (24 hour photoperiod) to prevent trapping of heat inside the cup. Mortality is assessed 3 days after treatment of the plants.

7. 2-Spotted Spider Mite (Tetranychus urticae, OP-Resistant Strain)

The active compounds are formulated in 50:50 acetone:water and 100 ppm Kinetic™ surfactant.

Sieva lima bean plants with primary leaves expanded to 7-12 cm are infested by placing on each a small piece from an infested leaf (with about 100 mites) taken from the main colony. This is done at about 2 hours before treatment to allow the mites to move over to the test plant to lay eggs. The piece of leaf used to transfer the mites is removed. The newly-infested plants are dipped in the test solution and allowed to dry. The test plants are kept under fluorescent light (24 hour photoperiod) at about 25° C. and 20-40% relative humidity. After 5 days, one leaf is removed and mortality counts are made.

8. Activity Against Cowpea Aphid (Aphis craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

In this test, the compound I-63 at 300 ppm showed a mortality of at least 75% in comparison with untreated controls.

9. Activity Against Diamond Back Moth (Plutella xylostella)

The active compounds were formulated in 50:50 acetone:water and 0.1% (vol/vol) Alkamuls EL 620 surfactant. A 6 cm leaf disk of cabbage leaves was dipped in the test solution for 3 seconds and allowed to air dry in a Petri plate lined with moist filter paper. The leaf disk was inoculated with 10 third instar larvae and kept at 25-27° C. and 50-60% humidity for 3 days. Mortality was assessed after 72 h of treatment.

In this test, the compounds I-63 and I-66 at 300 ppm showed a mortality of at least 75% in comparison with untreated controls.

10. Yellowfever Mosquitos (Aedes aegypti)

The test compound (1 Vol % in acetone) is applied to water in glass dishes containing 4th instar aedes aegypti. The test dishes are maintained at about 25° C. and observed daily for mortality. Each test is replicated in 3 test dishes.

11. Eastern Subterranean Termites (Reticulitérmes flávipes)

Toxicant treatments (1.0% test compound w/w) are applied to 4.25 cm (diam.) filter papers in acetone solution. Treatment levels (% test compound) are calculated on basis of a mean weight per filter paper of 106.5 mg. Treatment solutions are adjusted to provide the quantity of toxicant (mg) required per paper in 213 ml of acetone. Acetone only is applied for untreated controls. Treated papers are vented to evaporate the acetone, moistened with 0.25 ml water, and enclosed in 50×9 mm Petri dishes with tight-fit lids.

Termite bioassays are conducted in 100×15 mm Petri dishes with 10 g fine sand spread in a thin layer over the bottom of each dish. An additional 2.5 g sand is piled against the side of each dish. The sand is moistened with 2.8 ml water applied to the piled sand. Water is added to dishes as needed over the course of the bioassays to maintain high moisture content. Bioassays are done with one treated filter (inside enclosure) and 30 termite workers per test dish. Each treatment level is replicated in 2 test dishes. Test dishes are maintained at about 25° C. and 85% humidity for 12 days and observed daily for mortality.

12. Orchid thrips (Dichromothrips corbetti)

Dichromothrips corbetti adults used for bioassay are obtained from a colony maintained continuously under laboratory conditions. For testing purposes, the test compound is diluted to a concentration of 500 ppm (wt compound: vol diluent) in a 1:1 mixture of acetone:water, plus 0.01% Kinetic surfactant.

Thrips potency of each compound is evaluated by using a floral-immersion technique. Plastic petri dishes are used as test arenas. All petals of individual, intact orchid flowers are dipped into treatment solution for approximately 3 seconds and allowed to dry for 2 hours. Treated flowers are placed into individual petri dishes along with 10-15 adult thrips. The petri dishes are then covered with lids. All test arenas are held under continuous light and a temperature of about 28° C. for duration of the assay. After 4 days, the numbers of live thrips are counted on each flower, and along inner walls of each petri dish. The level of thrips mortality is extrapolated from pre-treatment thrips numbers.

### 13. Activity Against Argentine Ant, Harvester Ant, Acrobat Ant, Carpenter Ant, Fire Ant, House Fly, Stable Fly, Flesh Fly, Yellowfever Mosquito, House Mosquito, Malaria Mosquito, German Cockroach, Cat Flea, and Brown Dog Tick Via Glass Contact

Glass vials (20 ml scintillation vials) are treated with 0.5 ml of a solution of active ingredient in acetone. Each vial is rolled uncapped for ca. 10 minutes to allow the active ingredient to completely coat the vial and to allow for full drying of the acetone. Insects or ticks are placed into each vial. The vials are kept at 22° C. and are observed for treatment effects at various time intervals.

14. Activity Against Boll Weevil (Anthonomus grandis)

The active compounds were formulated in 1:3 DMSO:water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test, compounds I-31, I-61, I-63, I-65, I-66 and I-70 at 2500 ppm showed over 50% mortality.

15. Activity Against Mediterranean Fruitfly (Ceratitis capitata)

The active compounds were formulated in 1:3 DMSO:water. 50 to 80 eggs were placed into microtiterplates filled with 0.5% agar-agar and 14% diet in water. The eggs were sprayed with 5 μl of the test solution, the plates were sealed with pierced foils and kept at 27-29° C. and 75-85% humidity under fluorescent light for 6 days. Mortality was assessed on the basis of the agility of the hatched larvae. Tests were replicated 2 times.

In this test, compounds I-65 and I-66 at 2500 ppm showed over 50% mortality.

16. Activity Against Tobacco Budworm (Heliothis virescens)

The active compounds were formulated in 1:3 DMSO:water. 15 to 25 eggs were placed into microtiterplates filled with diet. The eggs were sprayed with 10 μl of the test solution, the plates were sealed with pierced foils and kept at 27-29° C. and 75-85% humidity under fluorescent light for 6 days. Mortality was assessed on the basis of the agility and of comparative feeding of the hatched larvae. Tests were replicated 2 times.

In this test, compounds I-64, I-69 and I-70 at 2500 ppm showed over 75% mortality.

17. Activity Against Vetch Aphid (Megoura viciae)

The active compounds were formulated in 1:3 DMSO:water. Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

In this test, compound I.66 at 2500 ppm showed over 75% mortality compared to 0% mortality of untreated controls.

1-30. (canceled)

31. A method for combating arthropodal pests, which comprises contacting said pests, their habitat, breeding ground, food supply, plant, seed, soil, area, material or environment in which the arthropodal pests are growing or may grow, or the materials, plants, seeds, soils, surfaces or spaces to be protected from an attack of or infestation by said pest, with a pesticidally effective amount of a N-(4-pyridyl)methylsulfonamide as defined by formula I 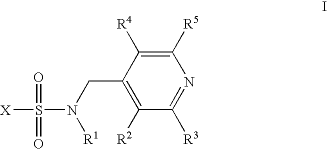
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′}, R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁₋haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I.

32. The method as claimed in claim 31, wherein at least one of the radicals R², R³, R⁴, or R⁵ is different from hydrogen.

33. The method as claimed in claim 31, wherein the radicals R², R³, R⁴, or R⁵ independently of one another are hydrogen, methyl, fluorine, chlorine, CF₃, OCF₃ or OCHF₂.

34. The method as claimed in claim 31, wherein the radicals R² and R³ together with the atoms to which they are attached form a condensed benzene ring, which optionally carries 1 or 2 radicals R^{2′} and/or R^{3′}.

35. The method as claimed in any of the preceding claims, wherein R¹ is hydrogen, methyl, methoxy, ethoxy, allyl, or propargyl.

36. The method as claimed in claim 35, wherein R¹ is hydrogen.

37. The method as claimed in claim 31, wherein X is a phenyl ring which is unsubstituted or carries 1, 2, or 3 radicals R^{a}.

38. The method as claimed in claim 37, wherein phenyl carries a radical R^{a} in the para position.

39. The method as claimed in claim 31, wherein X is an aromatic heterocycle.

40. The method as claimed in claim 39, wherein X is 2-thienyl, which is unsubstituted or which carries 1, 2, or 3 radicals R^{a} as defined in claim 31.

41. The method as claimed in claim 40, wherein X is 2-thienyl, which carries a radical R^{a} in the 5-position.

42. A compound of formula IA.4′ 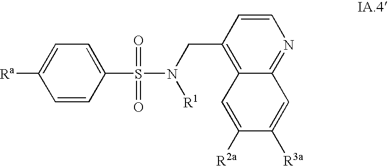
wherein R^{2a} and R^{3a} are both hydrogen, methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

43. A compound of formula IB 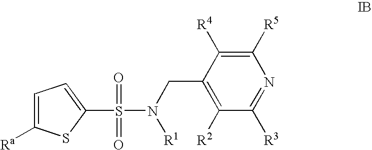
wherein R², R³, R⁴ and R⁵ are hydrogen, R¹ is hydrogen or methyl, and R^{a} is selected from the group consisting of phenyl, 4-phenyl-phenyl, 4-methyl-phenyl, and 5-ethyl-phenyl.

44. A compound of formula IB.1 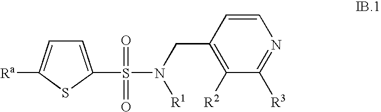
wherein R² and R³ are both methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

45. A compound of formula IB.2 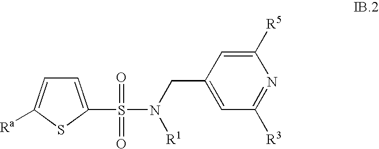
wherein R³ and R⁵ are both methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

46. A compound of formula IB.3 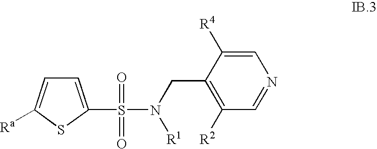
wherein R² and R⁴ are both methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

47. A compound of formula IB.4′ 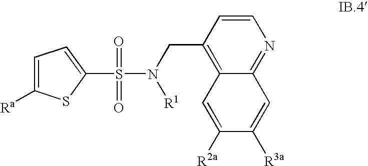
wherein R^{2a} and R^{3a} are both hydrogen, methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

48. A compound of formula IB.5 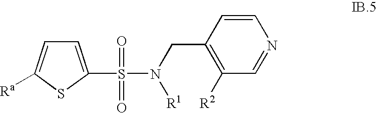
wherein R² is methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

49. A compound of formula IB.6 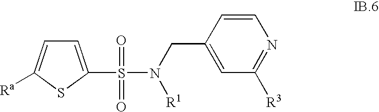
wherein R³ is methyl, fluorine, chlorine, methoxy, or trifluoromethoxy, R¹ is hydrogen or methyl, and R^{a} is selected from the group consisting of phenyl, 4-tert-butylphenyl, 4-phenyl-phenyl, 4-chloro-phenyl, 4-trifluoromethoxy-phenyl, 4-methoxy-phenyl, 4-trifluoromethyl-phenyl, 4-methyl-phenyl, 5-ethyl-phenyl, 4-(n-propyl)-phenyl, and 4-isopropyl-phenyl.

50. The method as claimed in claim 31, wherein the pests are insects.

51. The method as claimed in claim 31, wherein the pests are arachnids.

52. The method as claimed in claim 31, wherein at least one N-(4-pyridyl)methylsulfonamide as defined by formula I or a composition comprising it is applied in an amount of from 5 g/ha to 2000 g/ha.

53. A method for protecting crops from attack or infestation by arthropodal pests, the method comprising contacting a crop with a pesticidally effective amount of at least one N-(4-pyridyl)methylsulfonamide as defined by formula I 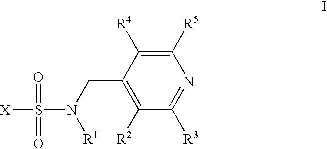
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′}, R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁-haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I.

54. The method as claimed in claim 53, wherein at least one N-(4-pyridyl)methylsulfonamide as defined by formula I or a composition comprising it is applied in an amount of from 5 g/ha to 2000 g/ha.

55. A method of protection of seed comprising contacting the seed with a N-(4-pyridyl)methylsulfonamide as defined by formula I 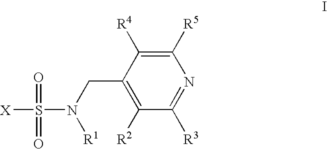
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′}, R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₉-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁-haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I, in pesticidally effective amounts.

56. A method as claimed in claim 55 wherein the N-(4-pyridyl)methylsulfonamide as defined by formula I or the composition comprising it is applied in an amount of from 0.1 g to 10 kg per 100 kg of seed.

57. Seed, comprising the N-(4-pyridyl)methylsulfonamide as defined by formula I 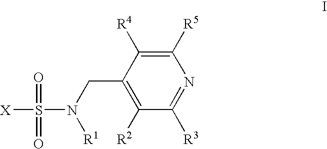
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′},R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁-haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I;
in an amount of from 0.1 g to 10 kg per 100 kg of seed.

58. A method for protecting non-living materials from attack or infestation by arthropodal pests, the method comprising contacting the non-living material with a pesticidally effective amount of at least one N-(4-pyridyl)methylsulfonamide I 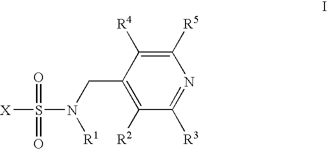
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′}, R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁-haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I.

59. A method for treating, controlling, preventing or protecting animals against infestation or infection by parasites which comprises orally, topically or parenterally administering or applying to the animals a parasiticidally effective amount of at least one N-(4-pyridyl)methylsulfonamide as defined by formula I 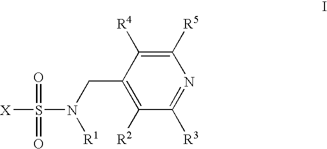
wherein:
R¹ is hydrogen, C₁-C₄-alkyl, C₁-C₄-alkoxy, C₂-C₄-alkenyl, C₂-C₄-alkynyl, or benzyl;
R², R³, R⁴, R⁵ independently of one another are hydrogen, halogen, C₁-C₄-alkyl, C₁-C₄-alkoxy, C₁-C₄-haloalkoxy, or C₁-C₄-haloalkyl;
R² and R³ or R⁴ and R⁵ together with the carbon atoms to which they are attached may also form a condensed 5- or 6-membered hydrocarbon ring, the hydrocarbon ring optionally having one or two groups R^{2′} and R^{3′},
R^{2′}, R^{3′} independently of one another are halogen, C₁-C₄-alkyl, C₁-C₄-alkoxy, halomethoxy, or halomethyl;
X is a cyclic radical selected from the group consisting of phenyl, naphthyl, and five- or six-membered saturated, partially unsaturated or aromatic heterocycles, the heterocycle being attached to the sulfur atom via a carbon atom and containing 1, 2, or 4 heteroatoms selected from the group consisting of the group consisting of O, N and S, wherein the cyclic radical X may carry 1, 2, 3, or 4 substituents R^{a}:
R^{a} is halogen, cyano, nitro, C₁-C₈-alkyl, C₁-C₈-haloalkyl, C₁-C₈-alkoxy, C₁-C₈-haloalkoxy, C₁-C₄-alkylcarbonyl, C₁-C₄-alkoxycarbonyl, —C(R⁶)═NOR⁷, C₁-C₄-alkylaminocarbonyl, di(C₁-C₄-alkyl)aminocarbonyl, or phenyl or phenoxy, wherein the phenyl ring in the last two mentioned radicals may carry 1, 2, 3, 4, or 5 groups R^{b}:
R⁶ is C₁-C₄-alkyl,
R⁷ is C₁-C₈-alkyl, benzyl, C₂-C₄-alkenyl, C₁-C₄-haloalkyl, C₂-C₄-haloalkenyl, C₂-C₄-alkynyl, or C₂-C₄-haloalkynyl; and
R^{b} is halogen, C₁-C₄-alkyl, C₁-C₄-alkoxy, C₁-haloalkyl, phenyl, optionally substituted with halogen, or haloalkoxy;
two radicals R^{a} or two radicals R^{b}, together with two adjacent ring members of the phenyl ring to which they are attached may form a hydrocarbon ring which may be substituted by one or more of the abovementioned groups R^{a} or R^{b},
with the exception of compounds wherein X and R^{a} together form an optionally substituted biphenyl, and R², R³, R⁴, and R⁵ independently of one another are hydrogen, halogen, C₁-C₄-alkyl, C₁-C₄-alkoxy, C₁-C₄-haloalkoxy, or C₁-C₄-haloalkyl;
or N-oxides or agriculturally and veterinarilly acceptable salts of compounds of formula I, or of compositions comprising compounds of formula I.
